(19) United States
(12) Patent Application Publication
(10) Pub. No.: US 2011/0165062 A1
(43) Pub. Date: Jul. 7, 2011

(54) Use of an anti-cd151 antibody for early treatment of cancer

(75) Inventor: Jean-Francois Haeuw, Beaumont (FR)

(73) Assignee: PIERRE FABRE MEDICAMENT, Boulogne (FR)

(21) Appl. No.: 12/736,358

(22) PCT Filed: Apr. 2, 2009
(86) PCT No.: PCT/FR2009/050561

§ 371 Date: Sep. 30, 2010

(30) Foreign Application Priority Data
Apr. 4, 2008 (FR) 0801865

(51) Int. Cl. A61K 51/00; A61K 39/395; C07K 16/32; A61P 35/00

(52) U.S. Cl. 424/1.11; 424/138.1; 530/387.7

(57) ABSTRACT

The present invention relates to the use of at least one antibody, or a functional fragment thereof, which is capable of binding to the CD151 protein and thereby inhibiting tumour growth, in the preparation of a medicament intended for the early treatment of cancer.

The invention is also directed to a composition for the treatment of cancer, comprising, as active ingredient, at least one anti-CD151 antibody, or a functional fragment thereof, which is capable of binding to the CD151 protein and/or inhibiting its metastasis-promoting activity, said antibodies consisting of antibodies secreted by the hybridoma deposited at the ATCC under number CRL-2696.

The present invention relates to a new use of anti-CD151 antibodies capable of inhibiting tumour growth, said antibodies being especially monoclonal of murine origin, chimaeric and humanised. According to a particular aspect, the invention relates to the use of those antibodies, or of functional fragments thereof, as medicaments for the early treatment of cancers and, very especially, of primary tumours. Finally, the invention includes products and/or compositions comprising such antibodies in association, for example, with anti-cancer agents and/or antibodies or conjugated with toxins, and use thereof in the prevention and/or treatment of certain cancers.

CD151, also referred to as PETA-3 or SFA-1, is a membrane protein belonging to the tetraspanin family (Boucheix and Rubinstein, 2001, Cell Mol. Life. Sci. 58, 1189-1205; Hemler, 2001, J. Cell Biol. 155, 1103-1107). In humans, CD151 has 253 amino acids and includes 4 membrane fragments and 2 extracellular domains EC1 (18 amino acids, sequence [40-57]) and EC2 (109 amino acids, sequence [113-221]) which are also referred to as extracellular loops. It is to be noted, however, that, in the nucleotide sequence, two variants of CD151 have been identified hitherto, namely one having nucleotides A and C at positions 395 and 409, respectively, [Fitter et al., 1995, Blood 86(4), 1348-1355] and the other having, at the same positions, nucleotides G and T instead of nucleotides A and C [Hasegawa et al., 1996, J. Virol. 70(5), 3258-3263]. As a result, a mutation can be observed in the peptide sequence, namely a mutation of the residues K (Lys) and P (Pro) at positions 132 and 137, respectively, to the residues R (Arg) and S (Ser) [Fitter et al., 1995, Blood 86(4), 1348-1355/Hasegawa et al., 1996, J. Virol. 70(5), 3258-3263].

CD151 is overexpressed in numerous cancers such as, for example, cancers of the lung [Tokuhara et al., 2001, Clin. Cancer Res. 7, 4109-4114], colon [Hashida et al., 2003, Br. J. Cancer 89, 158-167], prostate [Mg et al., 2004, Cancer Epidemiol. Biomarkers Prev. 13, 1717-1721] or pancreas [Gesierich et al., 2005, Clin. Cancer Res. 11, 2840-2852].

The use of knock-out mice which do not express CD151 and of anti-CD151 antibodies and siRNA in order to block, in vitro, the functionality and expression of CD151 in various types of cell has allowed it to be shown that CD151 is involved in a number of phenomena related to cancer, such as cell adhesion (Nishiuchi et al., 2005, Proc. Natl. Acad. Sci. USA 102, 1939-1944; Winterwood et al., 2006, Mol. Biol. Cell 17, 2707-2721), cell motility (Kohno et al., 2002, Int. J. Cancer 97, 336-343), cell migration (Yauch et al., 1998, Mol. Biol. Cell 9, 2751-2765; Testa et al., 1999, Cancer Res. 59, 3812-3820; Penas et al., 2000, J. Invest. Dermatol. 114, 1126-1135; Klosek et al., 2005, Biochem. Biophys. Res. Commun. 336, 408-416), cell invasion (Kohno et al., 2002, Int. J. Cancer 97, 336-343; Shiomi et al., 2005, Lab. Invest. 85, 1489-1506; Hong et al., 2006, J. Biol. Chem. 281, 24279-24292) and angiogenesis (Yanez-Mo et al., 1998, J. Cell Biol. 141, 791-804; Sincock et al., 1999, J. Cell Sci. 112, 833-844; Takeda et al., 2006, Blood).

One of the noteworthy properties of the tetraspanins is their ability to form associations amongst themselves and also with a large number of other surface molecules so as to form structured macromolecular complexes. Within those complexes, each tetraspanin is associated specifically with one or more surface molecules, thereby forming primary complexes composed of a tetraspanin and a partner molecule. The tetraspanins are capable of organising particular microdomains of the plasma membrane from which microdomains they may recruit their partner molecules, which may be functionally coupled. The set of interactions involving the tetraspanins has been referred to as the “network of tetraspanins” or “Tetraspanin Web”.

CD151 interacts on the surface of cells with various membrane proteins. In particular, there have been identified highly stable complexes, resistant to the action of certain detergents, with laminin receptor integrins, more particularly with the integrins α3β1 or α6β4, whose preferred ligand is laminin 5 (Yauch et al., 1998, Mol. Biol. Cell 9, 2751-2765; Lammerding et al., 2003, Proc. Natl. Acad. Sci. USA 100, 7616-7621). This association involves the extracellular domains of CD151 and the integrins. The sequence QRD [194-196] of CD151, located in the EC2 loop, is very important in that association because mutation of this site causes loss of interaction with certain integrins (Kazarov et al., 2002, J. Cell Biol. 158, 1299-1309). Functional ternary complexes of CD151/integrin α6β4/c-Met (HGF receptor) have moreover been identified in tumour cells (Klosek et al., 2005, Biochem. Biophys. Res. Commun. 336, 408-416). Inhibition of the expression of CD151 as a result of treating cells with an interference RNA results in inhibition of the cell growth and migration caused by HGF.

The interactions, within a particular cell, between CD151 and other tetraspanins, necessary for formation of the network of tetraspanins, are thought to depend on the membrane and cytoplasmic regions of CD151 because it has been shown that deletion of the EC2 loop does not disrupt the association of CD151 with other tetraspanins (Berditchevski, 2001, J. Cell Sci. 114, 4143-4151).

CD151 is capable of regulating the phenomena of cell adhesion, migration and invasion by modulation of various signalling pathways such as, for example, the phosphoinositide pathway via an association with PI4-kinase (Yauch et al., 1998, Mol. Biol. Cell 9, 2751-2765), the c-Jun signalling pathway via the phosphorylation de FAK, Src, p38-MAPK and JNK (Hong et al., 2006), the phosphorylation of integrins by PKC (Zhang et al., 2001, J. Biol. Chem. 276, 25005-25013) and the activation of GTPases of the Rho family (Shigeta et al., 2003, J. Cell Biol. 163, 165-176).

Homophilic-type interactions between cells are also responsible for an increase in cell motility and in expression of the metalloproteinase MMP-9 (Hong et al., 2006). Those intercellular CD151-CD151 interactions bring about the activation of c-Jun via the phosphorylation of FAK, Src, p38-MAPK and JNK.

Despite the interest in the CD151 protein, two therapeutically aimed antibodies have been generated to date, namely the monoclonal antibodies 50-6 and SFA1.2B4. These 2 antibodies have comparable activities. They do indeed inhibit the formation of metastases in vivo in animal models, but no effect on tumour growth in vivo has been demonstrated.

The monoclonal antibody 50-6 (IgG1 isotype) directed to CD151 was generated in the mouse by subtractive immunisations with human epidermoid carcinoma HEp-3 cells (Testa et al., 1999, Cancer Res. 59, 3812-3820).

The antibody 50-6 is capable of inhibiting, in vitro, migration of human cervical carcinoma HeLa cells, transfected so as to overexpress CD151, and of HEp-3 cells and angiogenesis in a model of chorio-allantoic membrane neovascularisation caused by bFGF (basic fibroblast growth factor). In vivo it inhibits the metastases brought about by inoculation of HEp-3 cells in 2 chicken embryo models (Testa et al., 1999, Cancer Res. 59, 3812-3820). In these models, the inhibitory activity of the antibody 50-6 is determined by measurement of the activity of the protein huPA (human urokinase-type plasminogen activator) in lung extracts. According to the authors, this assay reflects the presence of human cells in the lungs. After assaying, the reduction in metastases (dissemination of HEp-3 cells into the chicken embryo lungs) that is brought about by the antibody 50-6 is estimated, by comparison with a control antibody, to be 74% in a so-called “spontaneous metastasis” model, in which inoculation of the cells is followed by injection of the antibody, and 57% in a so-called “experimental metastasis” model, in which the cells and the antibody are inoculated together. According to the authors, the anti-tumour properties of the antibody 50-6 that are observed in vivo do not seem to be related to a cytostatic or cytotoxic effect because the antibody showed no effect on the in vitro proliferation of HEp-3 cells.

The hybridoma producing the antibody 50-6 is available at the ATCC under the to reference CRL-2696 (hybridoma initially deposited under the reference 50-6 [PTA-227]).

The anti-CD151 monoclonal antibody SFA1.2B4 (IgG1 isotype) was generated in the mouse after immunisation by the intraperitoneal route with NIH 3T3 cells transfected by the human CD151 gene (Hasegawa et al., 1996, J. Virol. 70, 3258-3263). The antibody SFA1.2B4 is capable of inhibiting in vitro cell invasion and motility of various human tumour lines (Kohno et al., 2002, Int. J. Cancer 97, 336-343). It inhibits in vivo the pulmonary metastases caused by colon cancer RPMI14788 and fibrosarcoma HT1080 lines transfected so as to overexpress CD151 (Kohno et al., 2002, Int. J. Cancer 97, 336-343).

Other murine anti-CD151 antibodies have been described in the literature, such as, for example, the monoclonal antibodies 14A2H1 (Ashman et al., 1991, Br. J. Haematol. 79, 263-270; Roberts et al., 1995, Br. J. Haematol. 89, 853-860), TS151 and TS151R (Serru et al., 1999, Biochem. J. 340, 103-111; Geary et al., 2001, Tissue Antigens 58, 141-153; Charrin et al., J. Biol. Chem. 276, 14329-14337; Chometon et al., 2006, Exp. Cell Res. 312, 983-985). No in vivo anti-tumour activity has been described for those various antibodies.

According to a first aspect, the invention is directed to use of a monoclonal antibody secreted by the hybridoma deposited at the ATCC under reference CRL-2696 or a functional fragment thereof, in the preparation of a medicament intended for inhibiting the tumour growth of a primary tumour for the early treatment of cancer. More specifically, said antibody is the monoclonal antibody 50-6. According to yet another aspect of the invention, the antibody whose use forms the subject-matter of the invention is specific for the CD151 protein.

Contrary to what had been known regarding said 50-6 antibody, the Applicant has demonstrated for the first time that this antibody has activity in inhibiting tumour growth and, very especially, growth of a primary tumour. Against all expectations, the invention accordingly relates to use of this antibody in preparing a pharmaceutical composition aimed at treating cancer early.

“Early treatment” or “treating early” is to be understood to refer to treatment inhibiting an early stage of the tumour development process before the appearance of metastases, as opposed to the later stages occurring once the metastatic process is under way. By way of non-limiting example, it may refer to inhibition of the proliferation of tumour cells within the primary tumour, that is to say prior to the metastatic process.

Several experimental studies have shown the major role of the tetraspanins in the formation of metastases by acting either as suppressors or as promoters of metastases. Accordingly, the transfection of tetraspanins such as CD9, CD63 or CD82 reduces the metastatic potential of cancer lines. In contrast, expression of the tetraspanins CD151 and Co-029 seems to produce the opposite effect. These 2 tetraspanins are therefore thought to be promoters of metastasis. These results are consistent with various clinical studies which have shown that, in a number of cancers (breast, lung, oesophagus, stomach, liver, pancreas, colon, prostate, melanoma . . . ), CD9 and CD82 are less expressed in primary tumours when there is metastasis and that a reduction in their expression is predictive of a lower survival rate. In lung cancer, the combined reduction in the expression of CD9 and CD82 has been correlated with a metastatic potential that is greater than when expression of just one of those two antigens is reduced.

Several retrospective studies have shown that overexpression of CD151 is associated with aggressiveness of certain cancers, such as lung, colon and prostate cancers, and that it might be considered to be a factor for poor prognosis (Tokuhara et al., 2001, Clin. Cancer Res. 7, 4109-4114; Hashida et al., 2003, Br. J. Cancer 89, 158-167; Ang et al., 2004, Cancer Epidemiol. Biomarkers Prey. 13, 1717-1721). In these cases, mean survival is in fact reduced in those patients having tumours which express CD151, compared to those having tumours which do not express CD151.

The overexpression of CD151 in various human tumour lines (HeLa, RPMI14788, A172, HT1080), brought about by transfection of the corresponding gene, causes an increase in the motility of, the migration of and invasion by the transfected cells (Testa et al., 1999, Cancer Res. 59, 3812-3820; Kohno et al., 2002, Int. J. Cancer 97, 336-343). These phenomena are inhibited in the presence of anti-CD151 antibodies.

According to another aspect, the functional fragments of antibodies according to the invention consist, for example, of Fv, scFv (sc standing for single chain), Fab, F(ab′)₂, Fab′ or scFv-Fc fragments or diabodies, or any fragment whose half-life may have been extended by chemical modification, e.g. addition of poly(alkylene)glycol such as poly(ethylene)glycol (“PEGylation”) (the PEGylated fragments being referred to as Fv-PEG, scFv-PEG, Fab-PEG, F(ab′)₂-PEG or Fab′-PEG) (“PEG” from the designation Poly(Ethylene)Glycol), or by incorporation in a liposome, microspheres or PLGA, said fragments being capable of generally exerting activity, even partial, of the antibody from which it is derived.

Preferably, said functional fragments will be composed of or will comprise a partial sequence of the variable heavy or light chain of the antibody from which they are derived, said partial sequence being sufficient to retain the same binding specificity as the antibody from which it is derived and an adequate affinity, preferably equal to at least 1/100th, more preferably at least 1/10th, of that of the antibody from which it is derived.

Such a functional fragment will comprise at least 5 consecutive amino acids, preferably 10, 15, 25, 50 or 100 consecutive amino acids, from the sequence of the antibody from which it is derived.

Preferably, these functional fragments will be fragments of Fv, scFv, Fab, F(ab′)₂, F(ab′), scFv-Fc type, or diabodies, which generally have the same fixing specificity as the antibody from which they are obtained. According to the present invention, fragments of antibodies of the invention can be obtained starting from antibodies as described hereinbefore by methods such as digestion using enzymes such as pepsin or papain and/or by cleavage of the disulfide bridges by means of chemical reduction. The antibody fragments included in the present invention can also be obtained by genetic recombination techniques that are likewise well-known to the person skilled in the art or by peptide synthesis by means of, for example, automatic peptide synthesisers such as those supplied by the company Applied.

According to an aspect of the invention, the antibody used consists of a murine monoclonal antibody.

Antibodies according to the present invention also include chimaeric or humanised antibodies.

A chimaeric antibody is understood as referring to an antibody which contains a natural variable (light chain and heavy chain) region derived from an antibody from a given species in association with the constant light chain and heavy chain regions of an antibody from a heterologous species to said given species.

Chimaeric-type antibodies, or their fragments, used in accordance with the invention can be prepared using genetic recombination techniques. For example, the chimaeric antibody may be produced by cloning a recombinant DNA comprising a promoter and a sequence coding for the variable region of a non-human, especially murine, monoclonal antibody according to the invention and a sequence coding for the constant region of a human antibody. A chimaeric antibody of the invention encoded by such a recombinant gene may be, for example, a mouse-human chimaera, the specificity of that antibody being determined by the variable region derived from the murine DNA and its isotype determined by the constant region derived from the human DNA. For methods of preparing chimaeric antibodies, reference may be made, for example, to the document Verhoeyn et al. (BioEssays, 8:74, 1988).

A humanised antibody is understood as referring to an antibody which contains CDR regions derived from an antibody of non-human origin, the other parts of the antibody molecule being derived from one (or more) human antibody/antibodies. In addition, some of the residues of the segments of the skeleton (referred to as FR) can be modified in order to preserve the binding affinity (Jones et al., Nature, 321:522-525, 1986; Verhoeyen et al., Science, 239:1534-1536, 1988; Riechmann et al., Nature, 332:323-327, 1988).

The humanised antibodies or functional fragments thereof can be prepared by techniques known to the person skilled in the art (such as, for example, those described in the documents Singer et al., J. Immun. 150:2844-2857, 1992; Mountain et al., Biotechnol. Genet. Eng. Rev., 10:1-142, 1992; or Bebbington et al., Bio/Technology, 10:169-175, 1992). Such humanised antibodies are preferred for their use in in vivo prophylactic and/or therapeutic treatment methods. Other humanisation techniques are also known to the person skilled in the art, such as, for example, the technique of “CDR Grafting”, described by PDL, which is the subject-matter of patents EP 0 451 261, EP 0 682 040, EP 0 939 127, EP 0 566 647 or also U.S. Pat. No. 5,530,101, U.S. Pat. No. 6,180,370, U.S. Pat. No. 5,585,089 and U.S. Pat. No. 5,693,761. There may also be mentioned the U.S. Pat. Nos. 5,639,641 or also 6,054,297, 5,886,152 and 5,877,293.

More specifically, the Applicant is putting forward, without wishing to be bound by any such theory, that the use of anti-CD151 antibodies in the context of cancer treatment may be of value not only due to inhibition of angiogenesis but also and above all due to inhibition of the metastasis-promoting activity of CD151. Indeed, contrary to what had been known hitherto, namely that the antibody 50-6 has an effect on angiogenesis, migration and invasion, the use according to the invention is directed to the very phenomenon of the tumour growth of primary tumours (with the evident direct consequence of inhibition of the formation of metastases and not only of the metastatic process, and especially of cell adhesion, cell migration and/or cell invasion.

As has been mentioned hereinbefore, the CD151 protein belongs to the tetraspanin family and, by virtue thereof, has 2 extracellular domains EC1 (18 amino acids, sequence [40-57]) and EC2 (109 amino acids, sequence [113-221]), also referred to as extracellular loops.

According to the present invention, the antibodies used are capable of binding to at least one epitope located in the extracellular domain. Preferably, said antibody will become fixed to the EC1 and/or EC2 loops.

More particularly, according to a preferred embodiment of the invention, there is described the use of at least one anti-CD151 antibody, or a functional fragment thereof, which is capable of binding to an epitope included in the extracellular loop 1 (EC1) and/or 2 (EC2), preferably EC2, corresponding to the amino acids 40-57 and 113-221, respectively, of the CD151 protein.

The EC1 loop [40-57] contains 18 amino acids and has a theoretical weight of 2002.2 Da.

The EC2 loop [113-221] has an N-glycosylation site (residue Asn159) and 6 cysteine residues forming 3 disulfide bridges. A structural model of the EC2 loop of the tetraspanins, and especially of CD151, has been proposed on the basis of the three-dimensional structure of the EC2 loop of the tetraspanin CD81 (Seigneuret et al., 2001, J. Biol. Chem. 276, 40055-40064). According to that model, the tetraspanins have a common, relatively conserved scaffold composed of 3 α helices and a specific variable domain. For CD151, that scaffold is thought to be composed of the regions [113-157] and [209-221], and the variable domain is thought to be composed of the region [158-208].

The variable domain of the EC2 loop is thought to be more especially involved in the specific interactions of CD151 with proteins of the integrin family. Directed mutagenesis experiments have especially shown the importance of the region [193-208], and more precisely of the tripeptide QRD [194-196] and the cysteine residue at position 192, in the association of CD151 with certain laminin receptor integrins such as integrins α3β1 or α6β4 (Kazarov et al., 2002, J. Cell Biol. 158, 1299-1309).

A “monoclonal antibody” is to be understood as an antibody derived from a population of substantially homogeneous antibodies. More especially, the individual antibodies of a population are identical with the exception of a few possible mutations that may be produced naturally and that may be present in minimal amounts. In other words, a monoclonal antibody consists of a homogeneous antibody resulting from the proliferation of just one cell clone (for example, a hybridoma, a eukaryotic host cell transfected with a DNA molecule coding for the homogeneous antibody, a prokaryotic host cell transfected with a DNA molecule coding for the homogeneous antibody etc.) and which is usually characterised by heavy chains of one and the same class and sub-class and light chains of just one type. Monoclonal antibodies are highly specific and are directed to a single antigen. In addition, in contrast to preparations of polyclonal antibodies which customarily comprise different antibodies directed to different determinants, or epitopes, each monoclonal antibody is directed to a single epitope of the antigen.

Preferably, use of the anti-CD151 antibodies in the context of cancer treatment is of value very especially in cancers overexpressing that same CD151 receptor.

Such cancers consist of colon cancer [Hashida et al., Br. J. Cancer 89 (2003):158-167], lung cancer, preferably non-small-cell lung cancer [Tokuhara et al., Clin. Cancer Res. 7 (2001):4109-4114], prostate cancer [Ang et al., Cancer Epidemiol. Biomarkers 13 (2004):17] and pancreatic cancer [Gesierich et al., Clin. Cancer Res. 11 [2005):2840-2852].

The present invention accordingly claims use of an antibody as described hereinbefore in the treatment of cancer, said cancer preferably consisting of colon, lung, prostate or pancreatic cancers.

The invention relates also to a pharmaceutical composition comprising, as active ingredient, a compound consisting of an antibody, or one of its derivative compounds or functional fragments, to which there is preferably added an excipient and/or a pharmaceutically acceptable carrier.

More especially, the invention is directed to use of an antibody according to the invention in the preparation of a pharmaceutical composition additionally comprising at least one pharmaceutically acceptable carrier.

In the present description, a pharmaceutically acceptable carrier is understood as referring to a compound or combination of compounds included in a pharmaceutical composition which does not give rise to secondary reactions and which, for example, makes it possible to facilitate the administration of the active compound(s), to increase the life and/or efficacy thereof in the body, to increase the solubility thereof in solution or to improve its storage. Such pharmaceutically acceptable carriers are well-known and will be adapted by the person skilled in the art as a function of the nature and mode of administration of the selected active compound(s).

Preferably, those compounds will be administered by a systemic route, especially the intravenous route, by the intramuscular, intradermal, intraperitoneal or subcutaneous route, or by the oral route. More preferably, the composition comprising the antibodies according to the invention will be administered on a plurality of occasions staggered over time.

Their optimal modes of administration, dosage regimens and galenic forms can be determined according to criteria generally taken into consideration in establishing a suitable treatment for a patient such as, for example, the age or bodyweight of the patient, the severity of his or her general condition, the tolerability of the treatment and the secondary effects established.

According to the invention there is described a composition for the treatment of cancer, characterised in that it comprises, as active ingredient, at least one monoclonal antibody secreted by the hybridoma deposited at the ATCC under reference CRL-2696, or a functional fragment thereof.

More especially, there is described a composition comprising at least one anti-CD151 antibody, or a functional fragment thereof, said monoclonal antibody consisting of the antibody 50-6.

The literature shows that the CD151 protein is overexpressed in cancers and, very especially, in colon carcinomas [Hashida et al., Br. J. Cancer 89 (2003): 158-167], non-small-cell lung cancers [Tokuhara et al., Clin. Cancer Res. 7 (2001): 4109-4114], prostate cancers [Ang et al., Cancer Epidemiol. Biomarkers 13 (2004): 1717-1721] and pancreatic cancers [Gesierich et al., Clin. Cancer Res. 11 (2005): 2840-2852].

Of course, the above list is given solely by way of illustration and any cancer must be understood as overexpressing the CD151 protein and therefore as being capable of being treated in accordance with the present invention. Another, complementary embodiment of the invention consists of a composition as described hereinbefore which additionally comprises a cytotoxic/cytostatic agent, as a combination product for simultaneous, separate or time-staggered use.

The present invention accordingly relates also to a composition as described hereinbefore, characterised in that it additionally comprises, as a combination product for simultaneous, separate or time-staggered use, at least one cytotoxic/cytostatic agent and/or a cell toxin and/or a radioelement.

“Simultaneous use” is understood as the administration of the two compounds of the composition according to the invention contained in one and the same pharmaceutical form.

“Separate use” is understood as the administration, at the same time, of the two compounds of the composition according to, the invention contained in separate pharmaceutical forms.

“Time-staggered use” is understood as the successive administration of the two compounds of the composition according to the invention, each contained in a separate pharmaceutical form.

In general manner, the composition according to the invention considerably increases the efficacy of the cancer treatment. In other words, the therapeutic effect of the antibody according to the invention is potentiated in unexpected manner by the administration of a cytotoxic agent. Another major subsequent advantage produced by a composition according to the invention relates to the possibility of using lower effective doses of active ingredient, which makes it possible to avoid or reduce the risks of secondary effects appearing, especially the effect of the cytotoxic agent. Moreover, this composition according to the invention should make it possible to achieve the expected therapeutic effect more rapidly.

“Anti-cancer therapeutic agents” or “cytotoxic agents” should be understood as substances which, when administered to a patient, treat or prevent the development of the cancer in the patient. By way of non-limiting example of such agents there may be mentioned “alkylating” agents, antimetabolites, anti-tumour antibiotics, mitotic inhibitors, chromatin function inhibitors, anti-angiogenesis agents, anti-oestrogens, anti-androgens or immunomodulators.

Such agents are, for example, mentioned in the VIDAL, on the page devoted to compounds used in oncology and haematology in the column “Cytotoxiques” (English: cytotoxic agents); such cytotoxic compounds mentioned by way of reference to that document are mentioned here as preferred cytotoxic agents.

“Alkylating agents” refer to any substance which is capable of covalently binding to or alkylating any molecule, preferably a nucleic acid (e.g.: DNA), within a to cell. As examples of such alkylating agents there may be mentioned nitrogen mustards such as mechlorethamine, chlorambucil, melphalan hydrochloride, pipobroman, prednimustine disodium phosphate or estramustine; oxazophorines such as cyclophosphamide, altretamine, trofosfamide, sulfofosfamide or ifosfamide; aziridines or ethylene-imines such as thiotepa, triethyleneamine or altetramine; nitrosoureas such as carmustine, streptozocin, fotemustine or lomustine; alkyl sulfonates such as busulfan, treosulfan or improsulfan; triazenes such as dacarbazine; and also platinum complexes such as cisplatin, oxaliplatin or carboplatin.

“Antimetabolites” refer to substances which block cell growth and/or cell metabolism by interfering with certain activities, generally DNA synthesis. By way of example of antimetabolites there may be mentioned methotrexate, 5-fluorouracil, floxuridine, 5-fluorodeoxyuridine, capecitabine, cytarabine, fludarabine, cytosine arabinoside, 6-mercaptopurine (6-MP), 6-thioguanine (6-TG), chlorodeoxyadenosine, 5-azacytidine, gemcitabine, cladribine, deoxycoformycin and pentostatin.

“Anti-tumour antibiotics” refer to compounds which can prevent or inhibit the synthesis of DNA, of RNA and/or of proteins. Examples of such anti-tumour antibiotics include doxorubicin, daunorubicin, idarubicin, valrubicin, mitoxantrone, dactinomycin, mithramycin, plicamycin, mitomycin C, bleomycin and procarbazine.

“Mitotic inhibitors” prevent the normal progression of the cell cycle and mitosis. In general, the microtubule inhibitors or “taxoids” such as paclitaxel and docetaxel are capable of inhibiting mitosis. The vinca alkaloids such as vinblastine, vincristine, vindesine and vinorelbine are also capable of inhibiting mitosis.

“Chromatin function inhibitors” or “topoisomerase inhibitors” refer to substances which inhibit the normal function of chromatin remodelling proteins such as topoisomerases I and II. Examples of such inhibitors include, for topoisomerase I, camptothecin and also its derivatives such as irinotecan or topotecan and, for topoisomerase II, etoposide, etiposide phosphate and teniposide.

“Anti-angiogenesis agents” refer to any drug, compound, substance or agent which inhibits the growth of blood vessels. Examples of anti-angiogenesis agents include, without any limitation, razoxin, marimastat, batimastat, prinomastat, tanomastat, ilomastat, CGS-27023A, halofuginone, COL-3, neovastat, BMS-275291, thalidomide, CDC 501, DMXAA, L-651582, squalamine, endostatin, SU5416, SU6668, interferon-alpha, EMD121974, interleukin-12, IM862, angiostatin and vitaxin.

“Anti-oestrogens” or “anti-oestrogen agents” refer to any substance which reduces, antagonises or inhibits the action of oestrogens. Examples of such agents are tamoxifen, toremifene, raloxifene, droloxifene, iodoxyfene, anastrozole, letrozole and exemestane.

“Anti-androgens” or “anti-androgen agents” refer to any substance which reduces, antagonises or inhibits the action of an androgen. Examples of anti-androgens are flutamide, nilutamide, bicalutamide, spironolactone, cyproterone acetate, finasteride and cimitidine.

Immunomodulators are substances which stimulate the immune system. Examples of such immunomodulators include interferons, interleukins such as aldesleukin, OCT-43, denileukin diflitox or interleukin-2, tumour necrosis factors such as tasonermin, or other types of immunomodulators such as lentinan, sizofuran, roquinimex, pidotimod, pegademase, thymopentin, poly I:C, or levamisole in combination with 5-fluorouracil.

For further details, the person skilled in the art will be able to refer to the manual published by the French Association of Teachers of Therapeutic Chemistry entitled “Traité de chimie thérapeutique, Vol. 6, Médicaments antitumoraux et perspectives dans le traitement des cancers, ed. TEC & DOC, 2003”.

In an especially preferred embodiment, said composition in the form of a combination product according to the invention is characterised in that said cytotoxic agent is chemically bound to said antibody for simultaneous use.

In an especially preferred embodiment, said composition according to the invention is characterised in that said cytotoxic/cytostatic agent is selected from spindle inhibitor or stabiliser agents, preferably vinorelbine and/or vinflunine and/or vincristine.

In order to facilitate binding between said cytotoxic agent and said antibody according to the invention, it will be possible, especially, to introduce spacer molecules between the two compounds to be bound, e.g. poly(alkylene)glycols such as polyethyleneglycol, or also amino acids, or, in another embodiment, to use active derivatives of said cytotoxic agents into which there will have been introduced functions capable of reacting with said antibody according to the invention. These binding techniques are well known to the person skilled in the art and will not be elaborated upon in the present description.

According to another aspect, the invention relates to a composition characterised in that one, at least, of said antibodies, or one of their derivative compounds or functional fragments, is conjugated with a cell toxin and/or a radioelement.

Preferably, said toxin or said radioelement is capable of preventing the growth or proliferation of the tumour cell, especially of totally inactivating said tumour cell.

Preference is also given to said toxin being an enterobacterial toxin, especially Pseudomonas exotoxin A.

The radioelements (or radioisotopes) employed in therapy, preferably conjugated with the antibody, are radioisotopes which emit gamma rays, preferably iodine¹³¹, yttrium⁹⁰, gold¹⁹⁹, palladium copper⁶⁷, bismuth²¹⁷ and antimony²¹¹. Radioisotopes which emit beta and alpha rays may also be used in therapy.

A toxin or radioelement conjugated with at least one antibody, or a functional fragment thereof, according to the invention is understood to refer to any means making it possible to bind said toxin or said radioelement to said at least one antibody, especially by covalent binding between the two compounds, with or without introduction of a linking molecule.

Among the agents allowing chemical (covalent), electrostatic or non-covalent linkage of all or some of the conjugate's elements there may be mentioned, very especially, benzoquinone, carbodiimide and, more especially, EDC (1-ethyl-3-[3-dimethylaminopropyl]-carbodiimide hydrochloride), dimaleimide, dithiobis-nitrobenzoic acid (DTNB), N-succinimidyl S-acetyl thioacetate (SATA), agents referred to as “bridging” agents having one or more groups, with one or more phenylazide groups, reacting with ultraviolet (UV) and very preferably N-[-4-(azidosalicylamino)butyl]-3′-(2′-pyridyldithio)propionamide (APDP), N-succinimidyl 3-(2-pyridyldithio)propionate (SPDP) and 6-hydrazino-nicotinamide (HYNIC).

Another form of binding, very especially for radioelements, can consist of using a bifunctional ion chelator.

Among those chelators there may be mentioned the chelators derived from EDTA (ethylenediaminetetraacetic acid) or DTPA (diethylenetriaminepentaacetic acid) that have been developed for binding metals, especially radioactive metals, and immunoglobulins. Accordingly, DTPA and its derivatives can be substituted with different groups on the carbon chain so as to increase the stability and rigidity of the ligand-metal complex (Krejcarek et al. (1977); Brechbiel et al. (1991); Gansow (1991); U.S. Pat. No. 4,831,175).

For example, DTPA (diethylenetriaminepentaacetic acid) and its derivatives, which has long been used very widely in medicine and biology either in its free form or in the form of a complex with a metal ion, has the noteworthy characteristic of forming stable chelates with metal ions and of being bound to proteins of therapeutic or diagnostic interest such as antibodies for the development of radioimmunoconjugates in cancer therapy (Meases et al., (1984); Gansow et al. (1990)).

The invention relates also to a composition comprising the antibody according to the invention, said composition being characterised in that it additionally comprises at least one second anti-tumour antibody.

The present invention additionally comprises use of the composition according to the invention in the preparation of a medicament.

The present invention is accordingly directed more especially to use of a composition as described hereinbefore in the preparation of a medicament intended for the treatment of cancer. Among the cancers which may be prevented and/or treated preference is given to colon, lung, prostate or pancreatic cancer.

The invention relates also to the use of an antibody according to the invention in the preparation of a medicament intended for the specific targeting of a biologically active compound at cells expressing or overexpressing the CD151 receptor.

A biologically active compound is understood herein as referring to any compound capable of modifying, especially inhibiting, the activity of cells, especially their growth, their proliferation, or the transcription or translation of genes.

Other characteristics and advantages of the invention will emerge in the remainder of the description with the Examples and Figures, for which the legends are given hereinbelow.

### LEGENDS FOR FIGURES

FIG. 1 illustrates the in vivo anti-tumour activity of the antibody 50-6 in a prostate cancer xenograft model. PC3 cells were grafted into Swiss Nude mice (n=6) by the subcutaneous route. Five days after grafting of the cells, the mice receive, by the i.p. route, a challenge dose of 2 mg/mouse of the antibody under test followed by two administrations per week of a dose of 1 mg/mouse of this antibody. The tumour volume is evaluated by the formula π/6×length×width×thickness, and a Mann and Whitney test is carried out for statistical evaluation of the results.

FIG. 2 shows evaluation of the in vivo anti-tumour activity of the antibody TS151 in an orthotopic model of lung cancer. 1×10⁶ A549 cells are grafted into immunodepressed mice (n=10) by the intrapleural route. Seven days after grafting, the mice are treated, by the intraperitoneal route, with a challenge dose of 2 mg of TS151 antibody followed by treatment, twice a week for 5 weeks, with a dose of 1 mg of antibody per mouse. The control group is injected with PBS according to the same administration regimen.

FIG. 3 shows evaluation of the specificity of the antibody 50-6 by means of Western blot. After SDS-PAGE electrophoresis, the gel was stained with Coomassie blue (A) or the proteins were transferred onto nitrocellulose membranes in order to carry out a Western blot (B). In the latter case, the transfer membrane was then incubated with the antibody 50-6 (1 μg/ml) and then with a rabbit anti-mouse Ig polyclonal antibody coupled to peroxidase (GE Healthcare) before visualisation by chemoluminescence.

### EXAMPLE 1

### Effect of the Antibody 50-6 on In Vivo Growth of the PC3 Tumour Implanted Subcutaneously in the Nude Mouse

Given that overexpression of CD151 in prostate cancer tumour tissues had been observed by immunohistochemistry, evaluation of the anti-CD151 antibody 50-6 on a xenograft of PC3 prostate cancer cells was planned. The PC3 line is an androgen-independent prostate line obtained from the ATCC and cultured in F12K medium+10% FCS+L-Glutamine. For evaluation, 5×10⁶ PC3 cells are implanted in the right flank of Swiss Nude mice. Five days after implantation, the animals are randomised on the basis of tumour volume and assigned to 2 comparable groups. The tumour volume of the selected grafted animals is between 41 and 47 mm³ (volume calculated by the formula π/6×length×width×thickness) on day 0 of treatment. The animals are then given the purified antibody under test or PBS (control group) by the intra-peritoneal route. The antibody doses and the frequency of injections are as follows: challenge dose 2 mg/dose of antibody; maintenance dose 1 mg/dose twice a week.

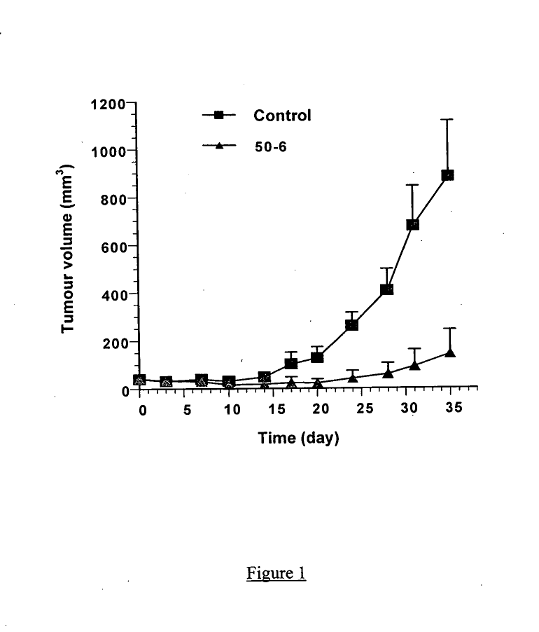

The results presented in FIG. 1 show that the antibody 50-6 significantly inhibits growth of the PC3 tumour implanted in a sub-cutaneous position in the Swiss Nude mouse. Statistical analysis shows that this inhibition of tumour growth is significant, compared to the control group, from day 10 of treatment (p<0.01).

### EXAMPLE 2

### Evaluation of the Anti-Tumour Activity of the Antibody 50-6 in an Orthotopic Model of Lung Cancer

A549 cells obtained from the ATCC are routinely cultured in F12K medium, 10 mM glutamine, 10% FCS. These cells are divided 2 days before grafting so that they will be in the exponential phase of growth. For grafting, 7-week-old immunodepressed mice are anaesthetised before being administered 1×10⁶ A549 cells by the intrapleural route. The primary tumour develops rapidly and in 4 days invades the structures adjacent to the injection site including the mediastinum, the lungs and the diaphragm. In order to mimic the disease better, starting treatment is not commenced until 7 days after implanting the cells, by the intraperitoneal route. After injection of a challenge dose of 2 mg/mouse, the antibody 50-6 is administered twice a week for 5 weeks, at a dose of 1 mg/mouse. A group of mice to which PBS is administered is introduced as a control, given that previously carried out experiments showed that administration of an IgG1 isotype control had no impact on the survival of the animals.

The evaluation parameter for this model is the survival of the animals, and the anti-tumour activity is expressed by calculation of the T/C %=median survival of the treated animals/median survival of the animals from the control group X 100. It has been established that a T/C % greater than or equal to 125% is indicative of activity of the product.

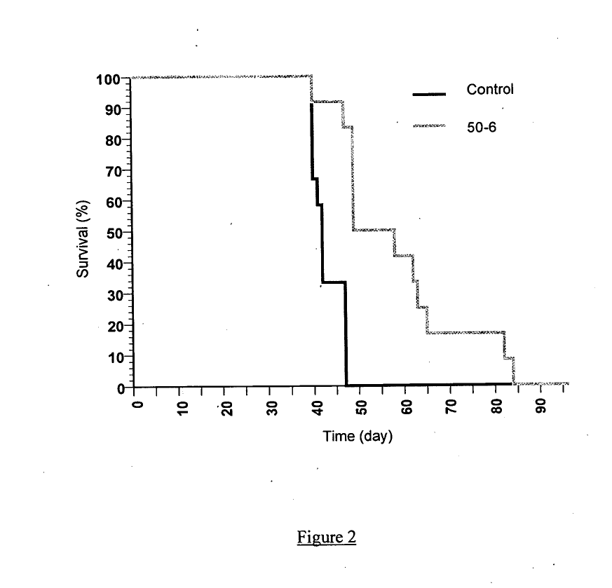

FIG. 2 shows anti-tumour activity of the antibody 50-6, with a calculated T/C % of 127% (p<0.001). This result confirms the anti-metastatic activity of the antibody 50-6.

### EXAMPLE 3

### Specificity of the Antibody 50-6

The specificity of the antibody 50-6 was evaluated by Western blot. The recombinant CD151 EC2 protein (5 μg) was placed on a 4-12% acrylamide gel (BioRad). After electrophoresis (non-reductive conditions), the proteins were transferred onto nitrocellulose membranes. The transfer membranes were then incubated with the purified 50-6 antibodies (1 μg/ml) and then with a rabbit anti-mouse Ig polyclonal antibody coupled to peroxidase (GE Healthcare) before visualisation by chemoluminescence.

The EC2 loop of CD151 was cloned into the vector pET22b for expression in soluble form in the periplasm of Escherichia coli. The protein that is produced includes amino acids 130 to 221 of the human CD151 peptide sequence to which there is added a Poly-His tail at the C-terminal position in order to facilitate purification. The recombinant EC2 protein was purified by immobilised metal affinity chromatography (IMAC) on a Chelating Sepharose HP support (GE Healthcare).

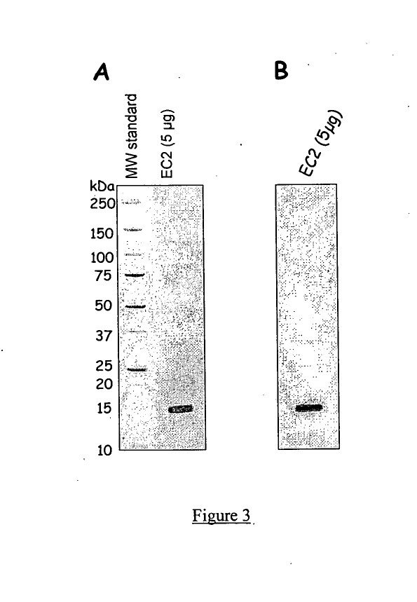

FIG. 3 shows that the anti-CD151 antibody 50-6 specifically recognises the EC2 loop by Western blot. This antibody is conformational because a loss of recognition is found when the Western blot is carried out after SDS-PAGE analysis under reductive conditions.

1-12. (canceled)

13. A method for the early treatment of cancer comprising administering to a patient in need thereof, a therapeutically effective amount of a monoclonal antibody secreted by a hybridoma deposited at the ATCC under reference CRL-2696, or a functional fragment thereof, wherein the monoclonal antibody inhibits the growth of a primary tumor.

14. The method of claim 13, wherein the monoclonal antibody is a 50-6 monoclonal antibody.

15. The method of claim 13, wherein the monoclonal antibody binds specifically to a CD151 protein.

16. The method of claim 15, wherein the antibody is capable of inhibiting the metastasis-promoting activity of said CD 151 protein within tumor cells.

17. The method of claim 13, wherein the cancer is selected from colon, lung, prostate and pancreatic cancer.

18. A pharmaceutical composition for the early treatment of cancer, comprising as active ingredient, at least one monoclonal antibody secreted by a hybridoma deposited at the ATCC under reference CRL-2696, or a functional fragment thereof.

19. The pharmaceutical composition of claim 18, wherein the monoclonal antibody is a 50-6 monoclonal antibody.

20. The pharmaceutical composition of claim 18, comprising at least one pharmaceutically acceptable carrier.

21. The pharmaceutical composition of claim 18, further comprising at least one cytotoxic/cytostatic agent and/or a cell toxin and/or a radioelement, in the form of a combination product.

22. The pharmaceutical composition of claim 21, wherein the at least one cytotoxic/cytostatic agent and/or a cell toxin and/or a radioelement is administered simultaneously, separately or time-staggered with the at least one monoclonal antibody secreted by a hybridoma deposited at the ATCC under reference CRL-2696, or a functional fragment thereof.

23. The pharmaceutical composition of claim 18, further comprising at least one second anti-tumor antibody.


SEQUENCE LISTING (6 sequences)

SEQ ID NO: 1 - DNA, 759 bp, Homo sapiens
atgggtgagttcaacgagaagaagacaacatgtggcaccgtttgcctcaagtacctgctgtttacctacaattgctgcttctggctggctggcctggctgtcatggcagtgggcatctggacgctggccctcaagagtgactacatcagcctgctggcctcaggcacctacctggccacagcctacatcctggtggtggcgggcactgtcgtcatggtgactggggtcttgggctgctgcgccaccttcaaggagcgtcggaacctgctgcgcctgtacttcatcctgctcctcatcatctttctgctggagatcatcgctggtatcctcgcctacgcctactaccagcagctgaacacggagctcaaggagaacctgaaggacaccatgaccaagcgctaccaccagccgggccatgaggctgtgaccagcgctgtggaccagctgcagcaggagttccactgctgtggcagcaacaactcacaggactggcgagacagtgagtggatccgctcacaggaggccggtggccgtgtggtcccagacagctgctgcaagacggtggtggctctttgtgggcagcgagaccatgcctccaacatctacaaggtggagggcggctgcatcaccaagttggagaccttcatccaggagcacctgagggtcattggggctgtggggatcggcattgcctgtgtgcaggtctttggcatgatcttcacgtgctgcctgtacaggagtctcaagctggagcactac

SEQ ID NO: 2 - protein, 253 aa, Homo sapiens
MGEFNEKKTTCGTVCLKYLLFTYNCCFWLAGLAVMAVGIWTLALKSDYISLLASGTYLATAYILVVAGTVVMVTGVLGCCATFKERRNLLRLYFILLLIIFLLEIIAGILAYAYYQQLNTELKENLKDTMTKRYHQPGHEAVTSAVDQLQQEFHCCGSNNSQDWRDSEWIRSQEAGGRVVPDSCCKTVVALCGQRDHASNIYKVEGGCITKLETFIQEHLRVIGAVGIGIACVQVFGMIFTCCLYRSLKLEHY

SEQ ID NO: 3 - DNA, 327 bp, Homo sapiens
gcctactaccagcagctgaacacggagctcaaggagaacctgaaggacaccatgaccaagcgctaccaccagccgggccatgaggctgtgaccagcgctgtggaccagctgcagcaggagttccactgctgtggcagcaacaactcacaggactggcgagacagtgagtggatccgctcacaggaggccggtggccgtgtggtcccagacagctgctgcaagacggtggtggctctttgtgggcagcgagaccatgcctccaacatctacaaggtggagggcggctgcatcaccaagttggagaccttcatccaggagcacctgagg

SEQ ID NO: 4 - protein, 109 aa, Homo sapiens
AYYQQLNTELKENLKDTMTKRYHQPGHEAVTSAVDQLQQEFHCCGSNNSQDWRDSEWIRSQEAGGRVVPDSCCKTVVALCGQRDHASNIYKVEGGCITKLETFIQEHLR

SEQ ID NO: 5 - DNA, 54 bp, Homo sapiens
tggacgctggccctcaagagtgactacatcagcctgctggcctcaggcacctac

SEQ ID NO: 6 - protein, 18 aa, Homo sapiens
WTLALKSDYISLLASGTY